(19) United States
(12) Patent Application Publication
(10) Pub. No.: US 2016/0161489 A1
(43) Pub. Date: Jun. 9, 2016

(54) Urine-based immuncassay for urocirtub 3 abd duagbisus if skeeo aobea

(71) Applicants: Mark W. Linder, Louisville, KY (US); Kevin Goudy, Decatur, GA (US); Roland Valdes, JR., Simpsonville, KY (US)

(72) Inventors: Mark W. Linder, Louisville, KY (US); Kevin Goudy, Decatur, GA (US); Roland Valdes, JR., Simpsonville, KY (US)

(21) Appl. No.: 14/756,545

(22) Filed: Sep. 15, 2015

Related U.S. Application Data
(60) Provisional application No. 62/050,750, filed on Sep. 15, 2014.

(51) Int. Cl. G01N 33/577; G01N 33/68

(52) U.S. Cl. CPC G01N 33/577; G01N 33/6893; G01N 2800/2864; G01N 2333/5751

(57) ABSTRACT

The present invention provides a method of identifying and isolating a set of monoclonal and polyclonal antibodies for use in immunometric assays for the detection of UCN III peptide, and the development of a two-site immunoassay for UCN III peptide. Monoclonal antibodies (mAb) have been affinity purified and used to develop a two-site immunometric assay comprising a mAb selected from the list comprising 2F7, 4E3, 4D3, 6D1, 1G10, 2E7, 4F3, 4C3, 5E11, 1A9, 4C4, 4B4, 2G7, 2A4, 1B9, 3H11, 5F2, 4G2, and 2E3 used for capture and a polyclonal antibody used for detection. The immunometric assay of the present invention comprise a method to identify and isolate specific antibodies or fragments thereof establishing the presence and/or amount of a UCN III peptide and detecting the specific binding necessary for determining a sample's UCN III peptide level for correlation with a diagnosis wherein the UCN III peptide level is used to determine whether the subject suffers from OSA.

### CROSS-REFERENCE TO RELATED APPLICATIONS

The present application claims priority from U.S. Provisional Application Ser. No. 62/050,750 filed on Sep. 15, 2014 and is incorporated by reference herein in its entirety.

### FIELD OF INVENTION

The present invention relates to a method of identifying and isolating each antigenic epitope for a UNC III peptide.

### BACKGROUND

The present development describes antibodies for use in immunoassays for detecting the Urocortin III peptide from a urine sample, and methods for using the antibodies in immunoassays for Urocortin-III.

Obstructive sleep apnea (OSA) is a condition that affects 2-III % of children in the United States. OSA is characterized by the partial or complete obstruction of the upper airway during sleep resulting in disruption in ventilation, hypoxia, and sleep fragmentation. Children suffering from OSA are more likely to develop behavior difficulties, learning disabilities, pulmonary hypertension, high blood pressure, cardiac dysfunction, systemic inflammation and decreased growth. OSA is normally diagnosed using polysomnography (an overnight sleep study), which is labor intensive, limited by availability and expensive.

Proteomic analysis of urine using mass-spectrometry has identified three proteins that are sensitive and specific for OSA: Urocortin-III peptide, Uromodulin peptide, and Orsomucoid peptide. Urocortin III peptide, (“UCN III”), is a 4-kilodalton peptide with a length of thirty-eight (III8) amino acids. UCN III peptide is expressed in kidney tubules, heart and brain, and the expression of UCN III peptide is induced by hypoxia. UCN III peptide has been found to be present in urine at a significantly higher level in children with OSA than in children who had not been diagnosed with OSA. UCN III peptide binds to the corticotropin-releasing factor receptor type II (CRFR2) and is involved in modulating stress responses, osmoregulation, and regulating the hypothalamus-pituitary-adrenal axis. UCN III peptide has also been found to correlate to the expression of CRFR2.

The prior art teaches a method for diagnosing obstructive sleep apnea (OSA) in a subject by determining the amount of one or more biomarkers in a biological sample provided from the subject. A method for diagnosing OSA consists of obtaining a biological sample from the subject; determining an amount in the sample of a Urocortin (UCN) III peptide; and comparing the amount of the UCN III peptide in the sample, if present, to a control level of the UCN III peptide. The subject is diagnosed as having OSA if there is a measurable difference in the amount of the UCN III peptide in the sample as compared to the control level. In some samples, the amount of a UNC III peptide is determined along with the amount of one or more peptides such as Uromodulin peptide, an Orosomucoid 1 peptide, and a Kallikrein1 peptide.

The amount of a UCN III peptide can be determined by using mass spectrometry analysis, immunoassay analysis, or both. The present comprises an immunoassay analysis performed by using an enzyme-linked immunosorbent assay (ELISA).

U.S. Pat. No. 8,999,658 for “Methods and Kits for Diagnosing Obstructive Sleep Apnea” is incorporated herein by reference in its entirety. U.S. Pat. No. 8,999,658 teaches that the presence or amount of a UCN III peptide can be determined using antibodies or fragments thereof specific for a UCN III peptide and detecting specific binding. The UCN III peptide biomarker may be captured by the use of immobilized antibodies or fragments thereof specific for UCN III peptide. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on a solid support. This strip can then be dipped into the test biological sample and then processed quickly through washes and detection steps to generate a measurable signal, such as for example a colored spot. The embodiments of the methods disclosed herein only call for a qualitative assessment of the presence or absence of UCN III peptide in the biological sample.

While the U.S. Pat. No. 8,999,658 teaches the presence or amount of a UCN III peptide can be determined using antibodies or fragments thereof specific for a UCN III peptide and detecting specific binding, the patent does not provide any specific antibodies that could be used effectively for this purpose.

Development of a simple, non-invasive, “dipstick” test to diagnose OSA using a urine biomarker, such as UCN III peptide, would be beneficial to the public by providing a relatively inexpensive and easily accessible diagnostic alternative to polysomnography. However, before this type of test can be developed, it is necessary to identify an immunometric assay which can effectively and efficiently detect the presence and quantity of UCN III peptide in a urine sample. The present development is the identification of suitable antibodies for use in an immunometric assay and the development of a two-site immunometric assay for the detection of UCN III peptide in urine specimens at concentrations greater than about 10 ng/mL.

The presently-disclosed subject matter provides methods and peptide biomarkers that can be utilized to diagnose OSA, and distinguish it from primary snoring without the need of an overnight sleep study, thereby providing earlier treatment opportunities. Methods for diagnosing OSA using peptide levels associated with OSA as biomarkers provides a means for determining whether to initiate or continue prophylaxis or treatment of OSA in a individual, by identifying at least one peptide biomarker associated with OSA in a biological sample.

A “biomarker” is a molecule useful as an indicator of a biologic state in an individual. The biomarker disclosed herein is a polypeptide (e.g., UCN III) that exhibits a change in expression or state, which can be correlated with the risk of developing, the presence of, or the progression of OSA in an individual. The peptide biomarker disclosed herein are inclusive of messenger RNAs (mRNAs) encoding the peptide biomarkers associated with OSA, as measurement of a change in expression of an mRNA can be correlated with changes in expression of the polypeptide encoded by the mRNA. As such, determining an amount of a peptide biomarker associated with OSA in a biological sample is inclusive of determining an amount of the peptide biomarker, including fragments thereof, and/or an amount of an mRNA encoding the peptide biomarker either by direct or indirect (e.g., by measure of a complementary DNA (cDNA) synthesized from the mRNA) measurement of the mRNA.

At least one peptide biomarker that is associated with OSA is a Urocortin (UCN) III peptide. The amount of a UCN III peptide in a biological sample is determined to diagnose OSA in an individual. A peptide biomarker of OSA for Urocortin III is GENBANK® Accession No. AAK67317 and SWISSPROT Identification Number Q969E3

Urocortin III peptides are 38 amino acid peptides which bind to the corticotropin-releasing factor receptor type II (CRFR2) typically induced by hypoxia and expressed in the renal bubules, cardiac tissue, systemic vasculature and the brain.

Associating a prognostic indicator with a predisposition to an adverse outcome is a statistical analysis. For example, a UCN III peptide level (e.g., quantity of expression in a sample) of greater than a control level in some embodiments can signal that an individual is more likely to suffer from OSA than individuals with a level less than or equal to the control level, as determined by a level of statistical significance. In some embodiments, a UCN III peptide level of less than or equal to a control level can signal that a subject does not suffer from OSA. Additionally, a change in UCN III peptide concentration from baseline levels can be reflective of subject prognosis, and the degree of change in marker level can be related to the severity of adverse events.

For example, a UCN III peptide level in a biological sample can be compared to a level known to be associated with OSA. The sample's UCN III peptide level is correlated with a diagnosis wherein the UCN III peptide level is used to determine whether the subject suffers from OSA, and respond accordingly. Alternatively, the sample's UCN III peptide level can be compared to a control UCN III peptide level known to be associated with a good outcome (e.g., the absence of OSA), such as an average level found in a population of normal subjects.

### SUMMARY OF THE INVENTION

The present invention comprises a method to identify and isolate specific antibodies or fragments thereof establishing the presence and/or amount of a UCN III peptide and detecting the specific binding necessary for determining a sample's UCN III peptide level for correlation with a diagnosis wherein the UCN III peptide level is used to determine whether the subject suffers from OSA.

A set of monoclonal and polyclonal antibodies is provided for use in immunometric assays for the detection of UCN III peptide, and the development of a two-site immunoassay for UCN III peptide. Monoclonal antibodies (mAb) have been affinity purified and used to develop a two-site immunometric assay comprising a mAb selected from the list comprising 2F7, 4EIII, 4DIII, 6D1, 1G10, 2E7, 4FIII, 4CIII, 5E11, 1A9, 4C4, 4B4, 2G7, 2A4, 1B9, IIIH11, 5F2, 4G2, and 2EIII used for capture and a polyclonal antibody used for detection.

The novelty of the instant assay is attributed to the method of identifying each antigenic epitope for UNC3 peptide wherein the UCN III peptide is divided into antigen epitope A and B. Each of the synthetic peptides are designed to target these different epitopes to generate unique, specific abs toward the antigenic determinant.

The novel method identifies the antibody clones that recognize each of the antigenic epitopes using the ELISA technique. The values in Table I are read in “OD values” resulting from the ab screening technique. The intensity of the OD values is an indirect measure of affinity of the antibodies. The antibodies can distinguish from A and B. Low binding affinity is indicative of non-specific antibodies.

One system for diagnosing OSA comprises means for obtaining a biological sample from a individual, means for determining the amount in the sample of a UCN III peptide, and means for comparing the amount of the UCN III peptide in the sample, if any, to a control level of the UCN III peptide to provide a diagnosis of OSA in an individual if there is a measurable difference in the amount of the UCN III peptide in the sample as compared to the control level. The biological sample may be selected from a urine sample, a saliva sample, a blood sample, a plasma sample, and a serum sample of an individual or sub-fractions thereof. The method of use may include a kit for diagnosing OSA includes an antibody capable of detecting a UCN III peptide and instructions for using the kit.

The present invention comprises or consists of a system for diagnosing OSA in a subject comprising the step of obtaining a biological sample from a subject, determining the amount in the sample of a UCN III peptide by identifying and isolating specific antibodies or fragments thereof establishing the presence and/or amount of a UCN III peptide and detecting the specific binding necessary for determining a sample's UCN III peptide level, and comparing the amount of a UCN III peptide in a sample to a control level of the UCN III peptide, determining if there is a measurable difference in the amount of the UCN III peptide in the sample as compared to a control level.

The present invention provides a method for diagnosing obstructive sleep apnea in a subject, comprising or consisting or providing a biological sample from the subject; determining an amount in the sample of a Urocortin III peptide; identify specific antibodies or fragments thereof establishing the presence and/or amount of a UCN III peptide; detecting the specific binding necessary for determining a sample's UCN III peptide level; isolating specific antibodies or fragments thereof establishing the presence and/or amount of a UCN III peptide; comparing the amount of the Urocortin III peptide in the sample, if present, to a control level of the Urocortin III peptide; and

diagnosing the subject as having obstructive sleep apnea, or a risk thereof by correlation of the amount of Urocotin III peptide with said control level of the UCN III peptide to determine whether the subject suffers from OSA.

More particularly, the present invention provides a method for the detection of UCN III peptide in urine comprising or consisting of providing a substrate for retaining a monoclonal antibody, wherein said substrate is suitable for exposure to urine including providing a monoclonal antibody and irreversibly affixing the monoclonal antibody to a substrate to create a test strip. A urine specimen is provided and exposed to the test strip whereby excess urine is removed from the test strip. A polyclonal antibody solution is provided for treating the urine-exposed test strip to determine if the polyclonal antibody is present on said test strip. The presence of polyclonal antibody is reported as a positive test for the presence of UCN III peptide and the absence of polyclonal antibody is reported as a negative test for the presence of UCN III peptide. The monoclonal antibody used in the method of UCN III peptide in urine is selected from the group consisting of 2F7, 4EIII, 4DIII, 6D1, 1G10, 2E7, 4FIII, 4CIII, 5E11, 1A9, 4C4, 4B4, 2G7, 2A4, 1B9, IIIH11, 5F2, 4G2, and 2EIII. The polyclonal antibody can comprise an anti-UCN III peptide. The step of biotinylating the polyclonal antibody is performed in order to determine the presence and/or quantity of UCN III peptide.

It is another object of the present invention to provide an immunoassay and method of use to labeled molecules in various sandwich, competitive, or non-competitive assay formats to generate a signal that is related to the presence or amount of an analyte of interest.

It is another object of the present invention to determine the presence of a UNC III peptide using specific antibodies or fragments thereof and detecting specific binding.

It is another object of the present invention to determine an antibody specifically binding UCN III which is inclusive of antibodies that bind the full-length peptide or a fragment thereof.

Other objects, features, and advantages of the invention will be apparent with the following detailed description taken in conjunction with the accompanying drawings showing a preferred embodiment of the invention.

### BRIEF DESCRIPTION OF THE DRAWINGS

A better understanding of the present invention will be had upon reference to the following description in conjunction with the accompanying drawings in which like numerals refer to like parts throughout the views wherein:

FIG. 1A-1D are graphs showing the diagnostic utility as set forth by the receiver operator curves (ROC) for Urocortin III (FIG. 1A), Orosomucoid 1 (FIG. 1B), Uromoduline (FIG. 1C), and a Combination for Urocortin III/Orosomucoid 1/Uromoduline (FIG. 1D);

FIG. 2 is a graph showing the absorbance of the mAB clones;

FIG. 3 is a graph showing the combined Mab reactivities;

FIG. 4, is a graph showing the combined MAb reactivities for Peptide A, Peptide B, and UCN III for the mAb clones as compared to a positive and negative control;

FIG. 5 shows a BIACORE analysis of mAbs to determine the optimal capture antibody using surface plasmon resonance;

FIG. 6 shows the SPR analysis of the mAb 1B;

FIG. 7 shows the mechanism for mAb 1B9 capture using an enzyme-linked immunosorbent assay (ELISA); and

FIG. 8 shows a standard curve for 1B9 capture with rabbit polyclonal for UCN III in urine.

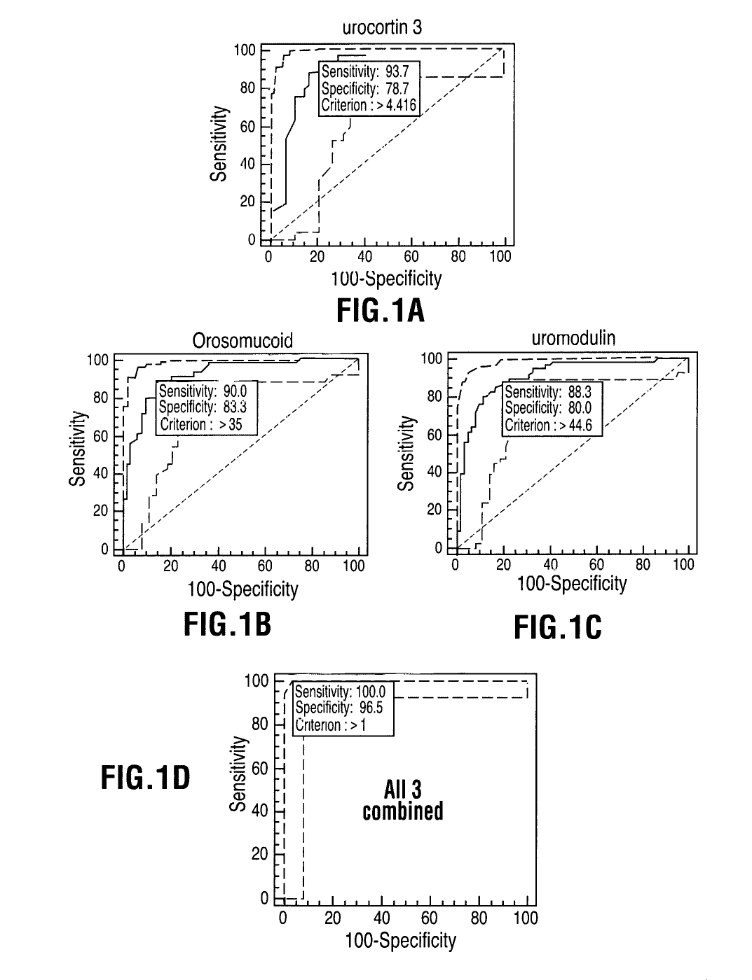

### DETAILED DESCRIPTION OF THE INVENTION

The following description is intended to provide the reader with a better understanding of the invention. The description is not intended to be limiting with respect to any element not otherwise limited within the claims. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Peptides described herein are described with reference to GenBank( ) accession numbers and SWISS-PROT identification numbers. The sequences cross-referenced in the GenBank® and SWISS-PROT databases are expressly incorporated by reference as are equivalent and related sequences present in GenBank®, SWISS-PROT, or other public databases. Also expressly incorporated herein by reference are all annotations present in the GenBank® and SWISS-PROT databases associated with the sequences disclosed herein. Unless otherwise indicated or apparent the references to the GenBank® database and the SWISS-PROT database are references to the most recent version of the database as of the filing date of this application.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

The terms “polypeptide”, “protein”, and “peptide”, which are used interchangeably herein, refer to a polymer of the 20 protein amino acids, including modified amino acids (e.g., phosphorylated, glycated, etc.) and amino acid analogs, regardless of size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein”, “polypeptide” and “peptide” are used interchangeably herein when referring to a gene product and fragments thereof. Exemplary polypeptides include gene products, naturally occurring proteins, homologs, orthologs, paralogs, fragments and other equivalents, variants, fragments, and analogs of the foregoing.

With regard to determining amounts of the UCN III peptide associated with OSA disclosed herein in samples, mass spectrometry, immunoassay devices and methods, and other methods are well known to those skilled in the art. Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule.

Any suitable immunoassay can be utilized, for example, enzyme-linked immunoassays (ELISA), radioimmunoassays (RIAs), competitive binding assays, immunoturbidimetric assays, and the like. Specific immunological binding of the antibody to the marker can be detected directly or indirectly. Direct labels include fluorescent or luminescent tags, metals, dyes, radionucleotides, and the like, attached to the antibody. Indirect labels include various enzymes well known in the art, such as alkaline phosphatase, horseradish peroxidase and the like.

The present invention comprises a method to identify and isolate specific antibodies or fragments thereof establishing the presence and/or amount of a UCN III peptide and detecting the specific binding necessary for determining a sample's UCN III peptide level

The present set of monoclonal antibodies for use in immunometric assays for the detection of UCN III peptide are prepared by immunization of C57BL6 mice with Peptide A (UCN III peptide amino acid sequence AA 1-25, N-terminal) conjugated with KLH or with Peptide B (UCN III peptide amino acid sequence AA 26-1118, C-terminal) conjugated with KLH. After immunization and cell fusion, the antibody-forming cells are then harvested and immortalized. Clones are subcultured and the supernatant is screened by ELISA with the microplate reader set at a wavelength of 450 mn to determine the highest reactivity to full-length UCN III peptide and to either the N-terminal or C-terminal fragments. The polyclonal antibodies are affinity purified.

The present set of polyclonal antibodies (pAb) for use in immunometric assays for the detection of UCN III peptide are prepared by immunization of New Zealand white rabbits with full-length KLH-conjugated UCN III peptide. After a fifty-five (55) day incubation period, the rabbits are bled and the pAb is affinity purified by techniques known to those skilled in the art.

The purified monoclonal antibodies (mAb) are used to develop a two-site immunometric assay. The immunoassay plates are coated with Peptide A, Peptide B or the full-length UCN III peptide (FL-UCN III peptide). Neat solutions of hybridoma supernatant were used as the primary antibody. Antibody pairs were identified using checkerboard ELISA and affinity characterization using BIACORE. Nineteen (19) hybridoma clones exhibited reactivity toward UCN III peptide. Specifically, the monoclonal antibodies 2F7, 4EIII, 4DIII, 6D1, 1G10, 2E7, 4FIII, 4CIII, 5E11, 1A9, 4C4, 4B4, 2G7, 2A4, 1B9, IIIH11, 5F2, 4G2, and 2EIII have been identified as demonstrating reactivity for unconjugated UCN III peptide. Five of these mAb clones, 4DIII, 2A4, 1B9, 5F2 and 4G2, exhibited absorbance greater than 1.0 to full-length UCN III peptide and Peptide A and/or Peptide B. In addition, noteworthy reactivity for full-length UCN III peptide and Peptide A and/or Peptide B was demonstrated by mAb clones 2E7, 2G7 and 5E11 in Table I.

TABLE I

| | Peptide | | |
|---|---|---|---|
| mAB | A | B | UCN III |
| 1A9 | 1.9975 | 0.III18 | 0.262 |
| 1B9 | 1.0765 | III.III68 | 2.8895 |
| 1G10 | 0.64 | 0.5655 | 0.429 |
| 2A4 | 1.8025 | 2.2805 | 1.III47 |
| 2EIII | 0.521 | 2.229 | 0.82III5 |
| 2E7 | 0.9875 | 1.0275 | 0.904 |
| 2F7 | 0.0765 | 0.08 | 0.072 |
| 2G7 | 1.184 | 1.287 | 0.8975 |
| IIIH11 | 0.8III45 | 1.2685 | 0.661 |
| 4B4 | 0.2745 | 0.III99 | 0.246 |
| 4CIII | 0.2945 | 0.III01 | 0.2III65 |
| 4C4 | 0.054 | 0.169 | 0.088 |
| 4DIII | 1.III705 | 1.IIIIII4 | 1.2215 |
| 4EIII | 0.IIIIII75 | 0.IIIIII7 | 0.III115 |
| 4FIII | 0.06III | 0.077 | 0.0685 |
| 4G2 | 1.959 | 1.96III5 | 1.599 |
| 5E11 | 1.III51 | 1.092 | 0.982 |
| 5F2 | 2.2225 | 2.IIIIII8 | 2.041 |
| 6D1 | 0.8095 | 2.5705 | 0.52III5 |

In the present development, the presence of UCN III peptide in a patient's urine is detected using an immunometric assay wherein the monoclonal antibody is used to capture the UCN III peptide present in the urine and then the polyclonal antibody is used to quantify the concentration of UCN III peptide in the specimen. For example, a substrate is coated with in Ab 4DIII; a urine specimen is flushed onto the 4DIII-coated substrate and excess urine is drained from the substrate; the polyclonal antibody anti-UCN III peptide is then flushed onto the 4DIII-coated substrate; the pAb-treated substrate is then biotinylated and detected using ELISA with the microplate reader set at a wavelength of 450 mn. The positive detection of the pAb anti-UCN III peptide is indicative of the presence of UCN III peptide in the urine, and the probability that the patient has obstructive sleep apnea. In the present example with mAb 4DIII and pAb anti-UCN III peptide, concentrations of UCN III peptide as low as 100 ng/mL have been detected, and an assay with a linear range from about 100 ng/mL to about III,100 ng/mL has been produced. The other monoclonal antibodies have demonstrated similar capture and detection reactivity to UCN III peptide in urine.

In order to run the assay for the detection of UCN III peptide in urine, it is anticipated that a test kit may be used. An exemplary test kit comprises: (a) a substrate carrying a monoclonal antibody; (b) a means for collecting a urine specimen; and © a polyclonal antibody source for treating the monoclonal antibody after it has been exposed to urine. In a preferred embodiment, the substrate is suitable for exposure to urine, and the monoclonal antibody is selected from the group consisting of 2F7, 4EIII, 4DIII, 6D1, 1G10, 2E7, 4FIII, 4CIII, 5E11, 1A9, 4C4, 4B4, 2G7, 2A4, 1B9, IIIH11, 5F2, 4G2, and 2EIII. The means for collecting a urine specimen may be any type of container known in the art for this purpose, such as a wide-mouth urine collection cup. The polyclonal antibody source is preferably anti-UCN III peptide. The components of the kit need not be available at a single location at a single point in time. For example, it would fall within the scope of the present invention to provide a patient with a urine-collection cup. The patient provides a urine specimen in the cup, and the urine specimen is transferred to a separate laboratory where the monoclonal antibody treated substrate is dipped into the urine specimen. The urine-exposed substrate may then be transferred to a different laboratory where the urine-exposed substrate will be treated with the polyclonal antibody, which may be further biotinylated, and the presence and/or quantity of UCN III peptide is determined using techniques known in the art.

It is anticipated that a simple, qualitative, non-invasive test method may be designed based on use of a monoclonal antibody for capture of UCN III peptide and a polyclonal antibody for detection of the captured UCN-III. In a preferred embodiment, the mAb is affixed to a suitable substrate, the mAb is then exposed to a urine specimen, excess urine is removed, the urine-exposed mAb is treated with a polyclonal antibody, and methods known in the art are used to detect the presence of the polyclonal antibody. The mAb is selected from the group consisting of 2F7, 4EIII, 4DIII, 6D1, 1G10, 2E7, 4FIII, 4CIII, 5E11, 1A9, 4C4, 4B4, 2G7, 2A4, 1B9, IIIH11, 5F2, 4G2, and 2EIII. In an exemplary embodiment, the captured UCN III peptide is detected via the use of a polyclonal antibody, such as anti-UCN III peptide.

It is further anticipated that a simple, quantitative, non-invasive test method maybe designed based on use of a monoclonal antibody for capture of UCN III peptide and a polyclonal antibody for detection of the captured UCN-III. In a preferred embodiment, excess mAb is affixed to a suitable substrate, the mAb is then exposed to a urine specimen, excess urine is removed, the urine-exposed mAb is treated with a polyclonal antibody, and methods known in the art are used to quantitatively assess the amount of polyclonal antibody present. For example, the amount of pAb present may be determined by measuring the fluorescence intensity at a wavelength of 450 urn and comparing the measured value to a previously generated calibration chart. Because there is a higher probability of obtaining accurate quantitative detection of the UCN III peptide if there is a strong affinity for the UCN III peptide by the mAb, the mAb is preferably selected from the group consisting of 2F7, 4EIII, 4DIII, 6D1, 1G10, 2E7, 4FIII, 4CIII, 5E11, 1A9, 4C4, 4B4, 2G7, 2A4, 1B9, IIIH11, 5F2, 4G2, and 2EIII. More preferably, the mAb is selected from the group consisting of 4DIII, 2A4, 1B9, 5F2, 4G2, 2E7, 2G7 and 5E11. Most preferably, the mAb is selected from the group consisting of 4DIII, 2A4, 1B9, 5F2 and 4G2. The pAb is most preferably anti-UCN III peptide.

Thus a set of monoclonal and polyclonal antibodies is provided for use in immunometric assays for the detection of UCN III peptide, and the development of a two-site immunoassay for UCN III peptide. Monoclonal antibodies (mAb) have been affinity purified and used to develop a two-site immunometric assay comprising a mAb selected from the list comprising 2F7, 4EIII, 4DIII, 6D1, 1G10, 2E7, 4FIII, 4CIII, 5E11, 1A9, 4C4, 4B4, 2G7, 2A4, 1B9, IIIH11, 5F2, 4G2, and 2EIII used for capture and a polyclonal antibody used for detection.

An example illustrating the technique and method of identifying and determining each antigenic epitope for UNC3 peptide wherein the UCN III peptide is divided into antigen epitope is set forth hereafter in Example 1.

### Example 1

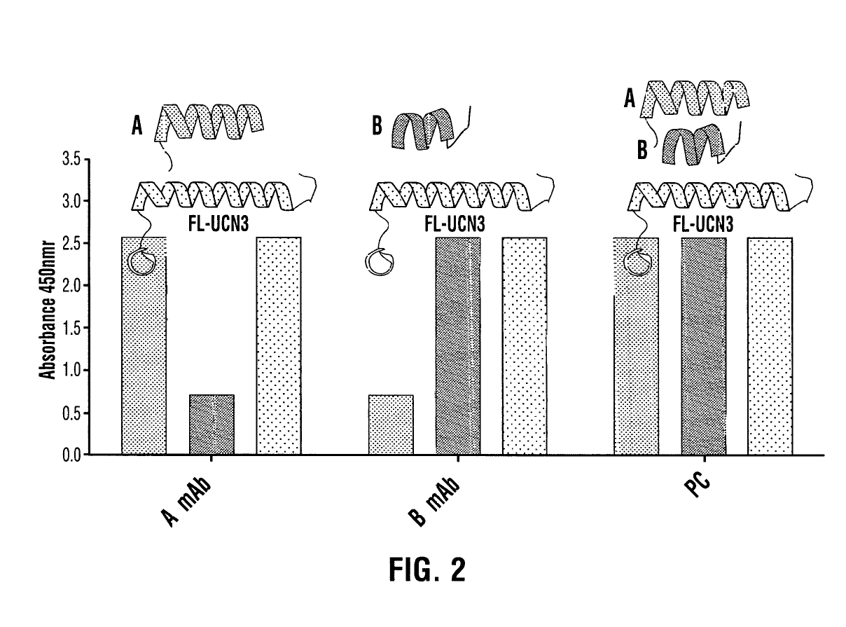

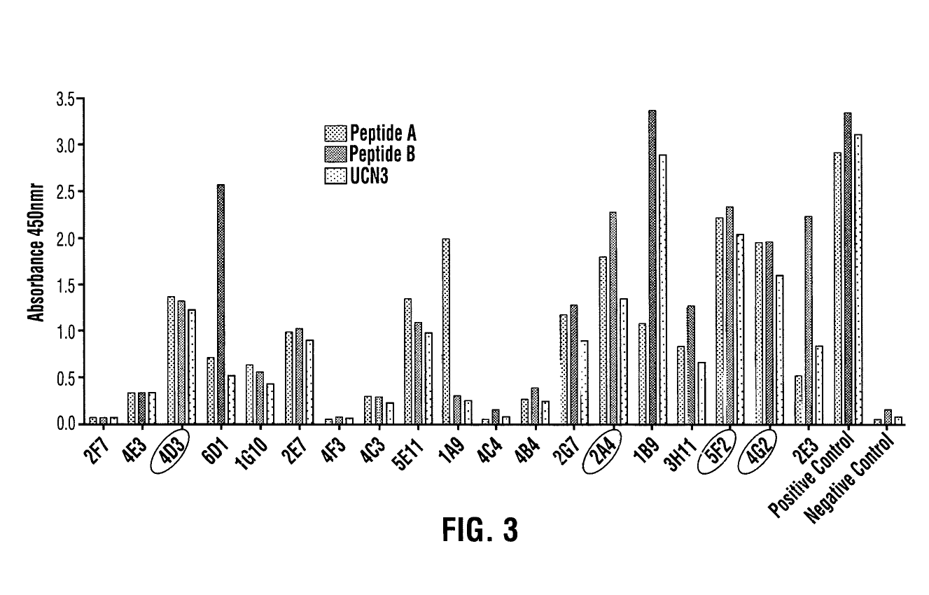

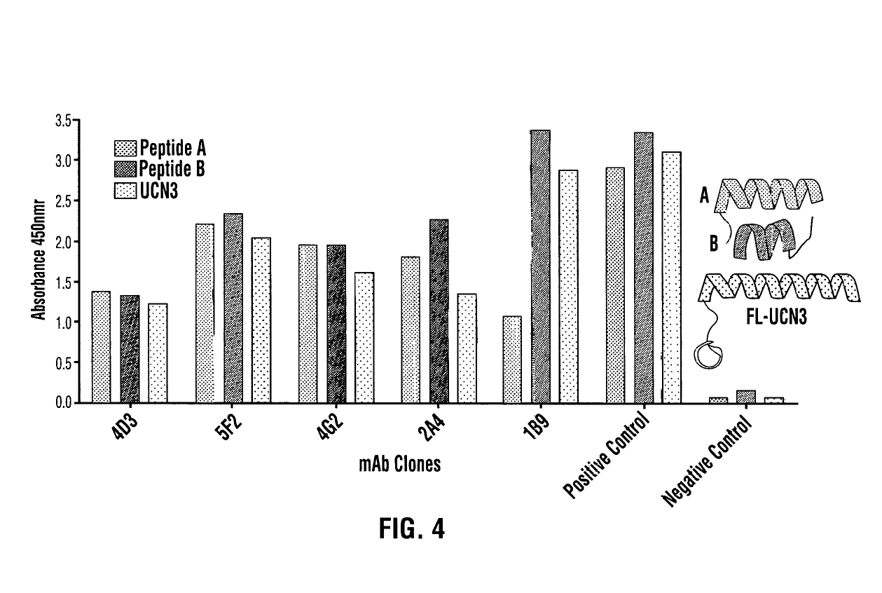

Development of the monoclonal antibodies comprised the following procedure. In silico analysis of the UCN III amino acid sequence was used to predict the immunogenic regions of the peptide. Two regions were identified. One near the N-term and one at the C-term. Mice were immunized with the two truncated peptides forming Peptides A and B for immunization and screening. Twenty clones were subcultured and supernatant was screened by ELISA to determine the highest reactivity to full length UCN III peptides. Plates were coated with Peptide A (1-25AA) UCNIII, Peptide B (26-38 UCN III), and FL UCN III. Neat solutions of hybridoma supernatant were used as the primary antibody. The mAB clones were screened and the absorbance determined as set forth in FIG. 2 and the combined MAb reactivities measured as set forth in FIG. 3 to determine positive or negative control. As shown in FIG. 4, the combined MAb reactivities for Peptide A, Peptide B, and UCN III is shown for the mAb clones as compared to a positive and negative control.

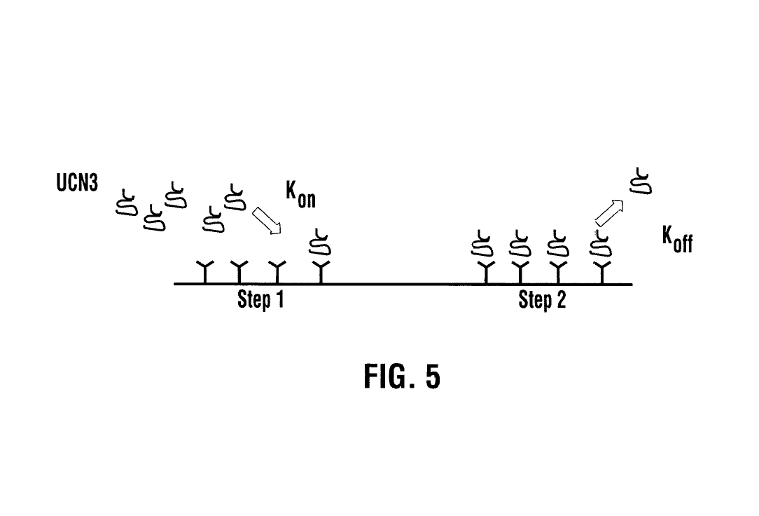

FIG. 5 shows a BIACORE analysis of mAbs to determine the optimal capture antibody using surface plasmon resonance, (SPR), which measures in real time the kinetics of protein-protein interactions and determines the binding kinetics (on/off raters) and dissociation constant K_{D}. More particularly, BIACORE is a company providing analytical services for measuring protein-protein interaction and binding affinity based on surface plasmon resonance, (SPR), an optical phenomenon that enables detection of unlabeled interactants in real time. The SPR-based biosensors can be used in determination of active concentration as well as characterization of molecular interactions in terms of both affinity and chemical kinetics. For example, a simple interaction experiment involves immobilizing one molecule of a binding pair on the sensor chip surface (“ligand”), and injecting a series of concentrations of its partner (“analyte”) across the surface. Changes in the index of refraction at the surface where the binding interaction occurs are detected by the hardware and recorded as RU (resonance units) in the control software.

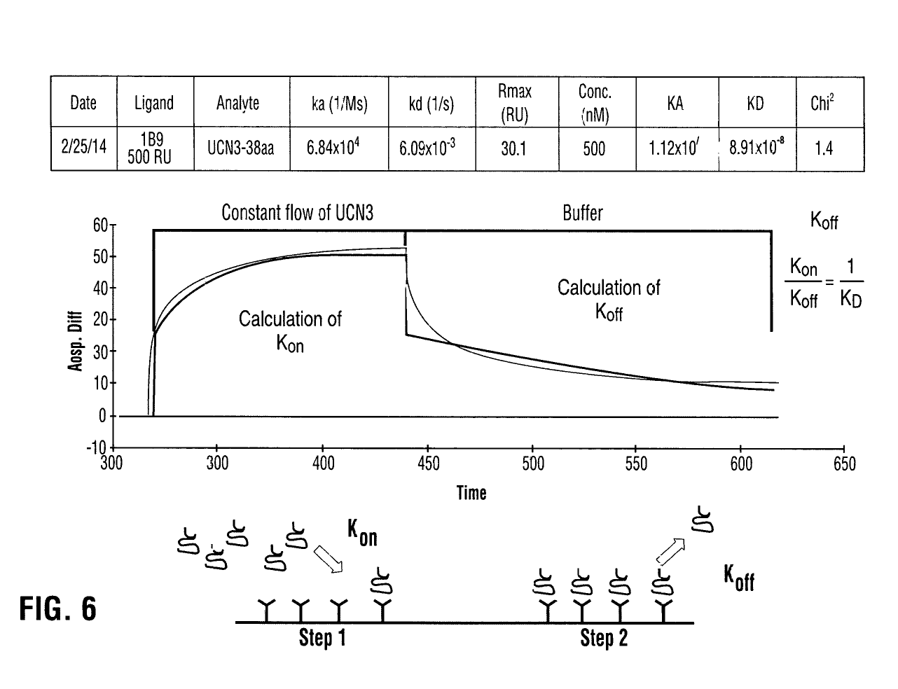

The SPR analysis of the mAb 1B is shown in FIG. 6, wherein curves are generated from the RU trace and are evaluated by fitting algorithms which compare the raw data to well-defined binding models. These fits allow determination of a variety of thermodynamic constants, including the apparent affinity of the binding interaction. SPR is one of many methods for protein-protein and protein-ligand interaction assessment.

The Mab screening with the SPR is shown in Table 2.

TABLE 2

| mAb Clone | Dissociation Constant (K_{D}) |
|---|---|
| 1B9 | 8.91 × 10⁻⁸M |
| 4D3 | 2.21 × 10⁻⁷M |
| 5F2 | 1.93 × 10⁻⁶M |
| 2A4 | 2.66 × 10⁻⁶M |
| 4G2 | 3.49 × 10⁻⁵M |
| Polyclonal Ab | 1.24 × 10⁻⁸M |

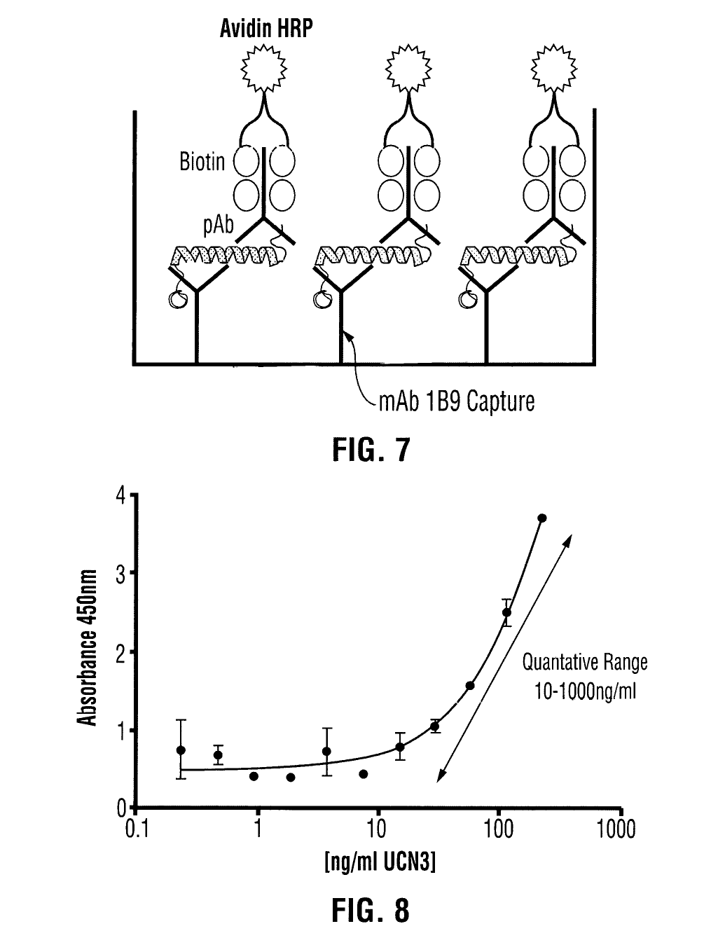

As shown in FIG. 7, an enzyme-linked immunosorbent assay (ELISA) was utilized with 96 well plates coated with capture antibody (mAb 1B9) wherein polyclonal Ab was used for detection of biotinylated and detection with streptavidin HRP. The UCN III peptide was tested in PBS and urine wherein the antibodies exhibit color change to identify a substance.

ELISA is a wet-lab type analytic biochemistry assay that uses a solid-phase enzyme immunoassay to detect the presence of an antigen, in a liquid sample or wet sample. Antigens from the sample are attached to a surface and a specific antibody is applied over the surface so it can bind to the antigen. This antibody is linked to an enzyme. A substance containing the enzyme's substrate is added and the subsequent reaction produces a detectable signal, most commonly a color change in the substrate. ELISA involves using at least one antibody with specificity for a particular antigen. The sample with an unknown amount of antigen is immobilized on a solid support such as a polystyrene microtiter plate either non-specifically (via adsorption to the surface) or specifically (via capture by another antibody specific to the same antigen, in a “sandwich”. After the antigen is immobilized, the detection antibody is added, forming a complex with the antigen. The detection antibody can be covalently linked to an enzyme, or can itself be detected by a secondary antibody that is linked to an enzyme through bioconjugation. Between each step, the plate is typically washed with a mild detergent solution to remove any proteins or antibodies that are non-specifically bound. After the final wash step, the plate is developed by adding an enzymatic substrate to produce a visible signal, which indicates the quantity of antigen in the sample. The technique essentially requires any ligating reagent that can be immobilized on the solid phase along with a detection reagent that will bind specifically and use an enzyme to generate a signal that can be properly quantified. In between the washes, only the ligand and its specific binding counterparts remain specifically bound or “immunosorbed” by antigen-antibody interactions to the solid phase, while the nonspecific or unbound components are washed away.

As shown in FIG. 8, a standard curve is depicted for the 1B9 capture with rabbit polyclonal is shown for UCN III in urine.

The foregoing detailed description is given primarily for clearness of understanding and no unnecessary limitations are to be understood therefrom, for modification will become obvious to those skilled in the art upon reading this disclosure and may be made upon departing from the spirit of the invention and scope of the appended claims. Accordingly, this invention is not intended to be limited by the specific exemplifications presented herein above. Rather, what is intended to be covered is within the spirit and scope of the appended claims.

We claim:

1. A set of monoclonal antibodies for use in immunometric assays for the detection of UCN III peptide in urine consisting of the monoclonal antibodies 2F7, 4EIII, 4DIII, 6D1, 1G10, 2E7, 4FIII, 4CIII, 5E11, 1A9, 4C4, 4B4, 2G7, 2A4, 1B9, IIIH11, 5F2, 4G2, and 2EIII.

2. A two-site immunometric assay for the detection of UCN III peptide in urine consisting of a monoclonal antibody used for capture of UCN III peptide and a polyclonal antibody used for detection of the captured UCN III peptide.

3. The assay of claim 2 wherein said monoclonal antibody is selected from the group consisting of 2F7, 4EIII, 4DIII, 6D1, 1G10, 2E7, 4FIII, 4CIII, 5E11, 1A9, 4C4, 4B4, 2G7, 2A4, 1B9, IIIH11, 5F2, 4G2, and 2EIII.

4. The assay of claim III wherein said monoclonal antibody is selected from the group consisting of 4DIII, 2A4, 1B9, 5F2 and 4G2.

5. The assay of claim 2 wherein said polyclonal antibody is anti-UCN III peptide.

6. A kit for a non-invasive test for the detection of UCN III peptide in urine comprising: a substrate for retaining a monoclonal antibody, wherein said substrate is suitable for exposure to urine and wherein said monoclonal antibody is selected from the group consisting of 2F7, 4EIII, 4DIII, 6D1, 1G10, 2E7, 4FIII, 4CIII, 5E11, 1A9, 4C4, 4B4, 2G7, 2A4, 1B9, IIIH11, 5F2, 4G2, and 2EIII;
a means for collecting a urine specimen;
a polyclonal antibody source for treating the monoclonal antibody after it has been exposed to urine.

7. The kit of claim 6 wherein said means for collecting a urine specimen is a wide-mouth urine collection cup.

8. The kit of claim 6 wherein said polyclonal antibody source is a solution comprising anti-UCN III peptide.

9. A method for the detection of UCN III peptide in urine comprising:
providing a substrate for retaining a monoclonal antibody, wherein said substrate is suitable for exposure to urine;
providing said monoclonal antibody
irreversibly affixing said monoclonal antibody to said substrate to create a test strip;
providing a urine specimen;
exposing said test strip to said urine;
removing excess urine from said test strip;
providing a polyclonal antibody solution;
treated said urine-exposed test strip with said polyclonal antibody solution; and,
determining if the polyclonal antibody is present on said test strip
wherein the presence of polyclonal antibody is reported as a positive test for the presence of UCN III peptide and the absence of polyclonal antibody is reported as a negative test for the presence of UCN III peptide.

10. The method of claim 9 wherein said monoclonal antibody is selected from the group consisting of 2F7, 4EIII, 4DIII, 6D1, 1G10, 2E7, 4FIII, 4CIII, 5E11, 1A9, 4C4, 4B4, 2G7, 2A4, 1B9, IIIH11, 5F2, 4G2, and 2EIII.

11. The method of claim 9 wherein said polyclonal antibody is anti-UCN III peptide.

12. The method of claim 9 further comprising the steps of:
biotinylating said polyclonal antibody; and
determining the presence and/or quantity of UCN III peptide.

13. The method of claim 12 wherein the presence and/or quantity of UCN III peptide is determined by measuring the quantity of polyclonal antibody present on said test strip using ELISA and comparing the result to a standardization curve.

14. A method for diagnosing obstructive sleep apnea in a subject consisting of:
a) providing a biological sample from the subject;
b) determining an amount in the sample of a Urocortin III peptide;
c) identify specific antibodies or fragments thereof establishing the presence and/or amount of a UCN III peptide;
d) detecting the specific binding necessary for determining a sample's UCN III peptide level;
e) isolating specific antibodies or fragments thereof establishing the presence and/or amount of a UCN III peptide;
f) comparing the amount of the Urocortin III peptide in the sample, if present, to a control level of the Urocortin III peptide; and
g) diagnosing the subject as having obstructive sleep apnea, or a risk thereof by correlation of the amount of Urocotin III peptide with said control level of the UCN III peptide to determine whether the subject suffers from OSA.
